(19) United States
(12) Patent Application Publication
(10) Pub. No.: US 2006/0084100 A1
(43) Pub. Date: Apr. 20, 2006

(54) Information nucleic acid and information nucleic acid composition using the same

(75) Inventors: Hiroshi Yokoyama, Kanagawa (JP); Masahiko Yamanaka, Kanagawa (JP); Kentarou Watanabe, Kanagawa (JP); Tsunehiko Higuchi, Nagoya (JP); Hidetoshi Hayashi, Nagoya (JP)

Correspondence Address: FOLEY AND LARDNER LLP;SUITE 500, 3000 K STREET NW, WASHINGTON, DC, 20007, US

(73) Assignees: Tsunehiko HIGUCHI; NISSAN MOTOR CO., LTD.

(21) Appl. No.: 11/239,465

(22) Filed: Sep. 30, 2005

(30) Foreign Application Priority Data
Sep. 30, 2004 (JP) 2004-286704

(51) Int. Cl. C12Q 1/68; C07H 21/04

(52) U.S. Cl. 435/6; 536/24.3

(57) ABSTRACT

An information nucleic acid includes a base sequence portion having an arbitrary and known base sequence. The information nucleic acid is mixed with an additive such as a fat solubilizing agent, a heat stabilizer, a light stabilizer, a nucleic acid hydrolase inhibitor and/or a dispersing agent so as to form an information nucleic acid composition.

### BACKGROUND OF THE INVENTION

This invention relates to improvements in an information nucleic acid and in an information nucleic acid composition using the information nucleic acid, and more particularly to the information nucleic acid and the information nucleic acid composition usable for individual authentication.

In order to authenticate an individuality of articles, a license plate, a watermarking for paper money, an IC chip, a facial portrait for a credit card and the like have hitherto been employed as an individuality authentication means.

However, these individuality authentication means have such drawbacks as to be able to be removed from a product, for example, by being peeled off, cut, erased. Accordingly, it has been desired to develop an authentication information which cannot be removed or erased from the product.

In this regard, DNA inherently contained in every organism is an information biopolymer including all genetic information of the organism. Most DNA correspond to many amino acid sequences of protein. DNA includes compounds such as deoxyadenosine (dA), deoxyguanosine (dG), deoxycytosine (dC) and thymidine (dT) which are bonded in a certain direction through phosphoric ester links. Assuming that the number of bases of DNA is n, 4ⁿ kinds of DNA will exist. Accordingly, the existence of about 4.3 billions kinds of distinguishable DNA is assumed even resulting from only 16 kinds of bases. At the present time, in synthesis of DNA having several tens of base sequences, any DNA having any base can be freely synthesized. In addition, concerning DNA in an amount more than a certain level, its base sequence can be automatically determined by an automatic sequence reader or sequencer.

With such a background, the following proposition has been made as disclosed in Japanese Patent Provisional Publication No. 2004-159502 in which a product is provided with a counterfeit-proof label made of a water-insoluble medium containing DNA. The authenticity of the product can be checked according to the presence or absence of DNA.

### SUMMARY OF THE INVENTION

However, the technique disclosed in Japanese Patent Provisional Publication No. 2004-159502 basically relates to a method for mixing DNA with the water-insoluble medium. As a method for checking the authenticity of the product, the publication discloses that the target product containing ribonucleic acid is identified by detecting whether ribonucleic acid is amplified or not by using a PCR method. Additionally, the publication does not disclose individuality authentication data using presence or absence of DNA as an examination index as well as data which relate to individuality authentication and makes possible the individuality authentication of each product even in the same kind of products.

By the way, in case where an article such as vehicle is stolen or vandalized by an assailant who has run away, it is required to specify the objective article as soon as possible according to pieces of paint or material of the article which pieces have been left at the crime scene.

In view of the above, an object of the present invention is to provide an improved information nucleic acid and information nucleic acid composition which can effectively overcome drawbacks encountered in conventional information nucleic acids and information nucleic acid compositions of similar nature.

Another object of the present invention is to provide an improved information nucleic acid and information nucleic acid composition with which the origins or histories of products can be specified to concretely and individually authenticate the products by causing the information nucleic acid and/or the information nucleic acid composition to have been contained in industrial products or the like.

An aspect of the present invention resides in an information nucleic acid which comprises a base sequence portion or site having an arbitrary and known base sequence.

Another aspect of the present invention resides in an information nucleic acid composition which comprises an information nucleic acid including a base sequence portion or site having an arbitrary and known base sequence. An additive includes at least one selected from the group consisting of a fat solubilizing agent, a heat stabilizer, a light stabilizer, a nucleic acid hydrolase inhibitor and a dispersing agent.

### BRIEF DESCRIPTION OF THE DRAWING

FIG. 1A is a structural formula of a natural type DNA;

FIG. 1B is a structural formula in which a hydroxyl group at position 5′ of DNA of FIG. 1A is derivatized; and

FIG. 2 is a schematic view showing the base sequence of a single-stranded DNA whose identification information site is provided with primer binding sites at its both ends.

### DETAILED DESCRIPTION OF THE INVENTION

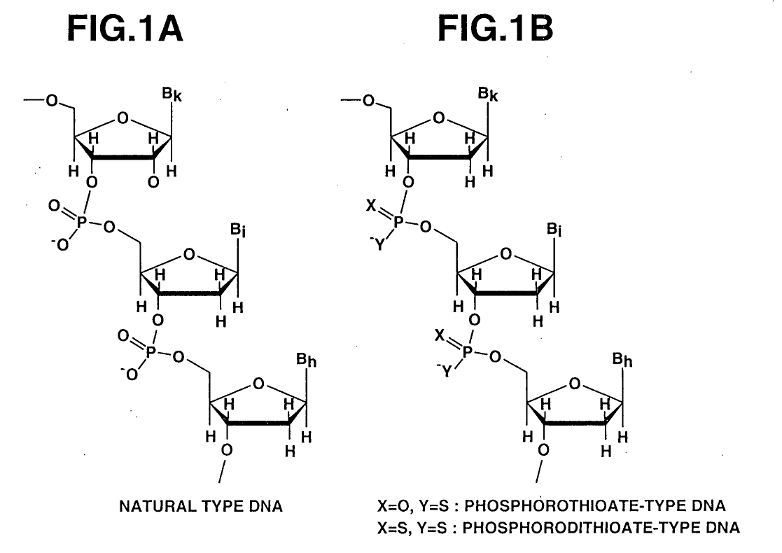

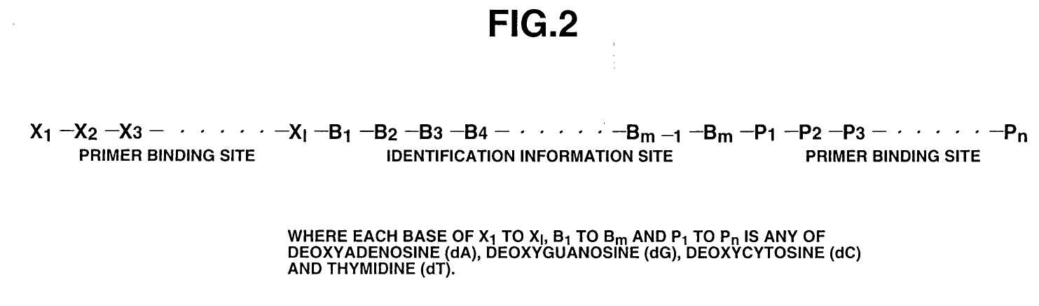

Referring now to FIGS. 1A, 1B and 2 of the drawings, an embodiment of an information nucleic acid according to the present invention will be discussed. In this specification, all percentages (%) are by mass unless otherwise specified.

The information nucleic acid according to the present invention includes a portion or site having an arbitrary and known base sequence which portion hereinafter referred to as a base sequence portion or site. The information nucleic acid can be easily contained in a product and the material of the product while being usable as an individuality authenticating means which is difficult to be removed from the product.

The information nucleic acid includes DNA (deoxyribonucleic acid), RNA (ribonucleic acid) and derivatives of DNA and RNA. Although either a natural type nucleic acid or an artificial type nucleic acid can be used, it is preferable to use the artificial type one which is structurally stable taking account of causing it to be contained in the product used under a severe condition. In the artificial type nucleic acid, the base sequence can be formed having bonding modes which do not exist in the natural type nucleic acid. In the bonding modes, a bond between nucleoside and nucleoside is not only a phosphoric ester link but also a nonnatural type one such as a thiophosphoric ester link.

Additionally, the arbitrary base sequence of the information nucleic acid means that the base sequence can be freely selected as far as it is detectable or readable. The known base sequence means that the base sequence used for individuality authentication has been previously grasped or ascertained.

With respect to size of the information nucleic acid, it is preferable that the number of bases in the whole of the information nucleic acid is not larger than 200. In case where the number of the bases is larger than 200, unreacted portions are made bit by bit at the stage of synthesis so that a content of nucleic acids whose bases are missed is liable to increase. It is more preferable that the number of the bases is about 100.

Further, it is preferable that thymine and thymine are not adjacent to each other in the above base sequence. This prevents dimerization of thymines. Further, it is preferable that the information nucleic acid is derivatized with a protective group from the view points of improving stability in cases where it is used together with a compound which can react with a hydroxyl group and used under the severe condition. Specifically, at least one of hydroxyl groups at positions 3′ and 5′ can be derivatized with a phosphoric ester group, an acyl group, an alkoxycarbonyl group, a benzyl group, a substituted benzyl group, an allyl group and the like. FIG. 1A shows a structural formula of a natural type DNA, and FIG. 1B shows a structural formula in which hydroxyl group at position 5′ of DNA illustrated in FIG. 1A is derivatized. In FIG. 1B, the illustrated DNA is of a phosphorothioate type in case that X is an oxygen atom and Y is a sulfur atom, and the illustrated DNA is of a phosphorodithioate type in case that both X and Y are sulfur atoms, respectively.

It is further preferable that the hydroxyl group at position 5′ is derivatized with biotin or fluorescent molecules from the viewpoint of improving the convenience in isolation and refinement of the information nucleic acid. Concretely, using biotin to derivative a part of the information nucleic acid facilitates a selective adsorption of the information nucleic acid to a column to which avidin (a kind of protein) is bonded. On the other hand, using fluorescent molecules such as fluorescein facilitates refinement and the like of the information nucleic acid since nucleic acid itself becomes fluorescent so as to be sensitively detectable. Thus, the improved convenience in isolation and refinement of the information nucleic acid largely facilitates the individuality authentication.

It will be understood that a hydroxyl group at position 2′ may be derivatized with the above-mentioned protective group from the viewpoint of improving stability, when RNA is used as the information nucleic acid.

Furthermore, when the individuality authentication is conducted on the information nucleic acid in a state of being contained in the product, it is preferable that a portion or site used for amplification of the information nucleic acid is the above-mentioned base sequence portion from the viewpoint of achieving an effective detection even in a low content of the information nucleic acid. As a method of the amplification of the information nucleic acid low in content, a polymerase chain reaction (PCR) by which DNA is synergistically amplified can be suitably employed.

Typically, it is preferable to use a PCR method using PCR by which the information nucleic acid even in a very small quantity can be highly amplified. With this PCR method, for example, by acting heat-resistance DNA polymerase on original DNA in the presence of bases or primers complementary to several tens of bases of the original DNA under a temperature control, the original DNA can be amplified. When this operation of amplification is repeated 30 times, the original DNA can be amplified several hundreds of millions times. Such amplification can provide a sufficient amount of DNA to determine the base sequence. As a result, the identity of the product containing the original DNA can be authenticated from the information corresponding the base sequence.

Additionally, in connection with the above, it is preferable that the original DNA has portions (primer binding sites) corresponding to primers at its both ends, as the above-mentioned portion used for the above amplification. The information nucleic acid which does not have the primer may be used; however, provision of the primer can make possible authentication of the original DNA within a short time.

Regarding the primer binding site, it is preferable that the number of the bases is not less than 5, and it is more preferable that the number of the bases is not less than 10. If the number of the bases is less than 5, the number of nucleic acids which are distinguishable is reduced, and therefore much time is necessary to distinguish a lot of target products individually. It is further preferable that the number of the bases is not larger than 100. If the number of the bases is larger than 100, the ratio of by-product missing a base at any position is unavoidably increased. Accordingly, it will take much time and effort to refine, and refinement will become difficult to be done in certain circumstances.

When RNA is used as the information nucleic acid, first DNA complementary in base sequence to the RNA is obtained by using reverse transcriptase, and thereafter the PCR method is carried out to accomplish amplification of the information nucleic acid.

Moreover, it is preferable that the information nucleic acid has an identification information site in addition to the above-mentioned base sequence portion. With this, more detailed information can be set, thereby accomplishing more advanced individual authentication. This is, for example, explained with reference to FIG. 2. As shown in FIG. 2, in case of an information DNA which has the primer binding sites at its both ends, m (number) base sequences (B₁ to Bₘ) are located at the middle, in which the sequence information of this m base sequences correspond to the identification information site. The primer binding sites having 1 (number) and n (number) base sequences (X₁ to X) and (P₁ to Pₙ) complementary respectively to 1 and n primers are connected to the both ends. Upon existence of this complementary base sequences, using the PCR method becomes possible for the first time. Either a single-stranded or a double-stranded information DNA can be used as an information element. The double-stranded information DNA is a complex of the single-stranded information DNA and the complementary DNA. The base sequence of the primer binding site can be arranged such that bonds to the complementary base sequence can be stable as much as possible and that the amplification by the PCR method can progress smoothly.

Next, details of an information nucleic acid composition according to the present invention will be discussed.

The information nucleic acid composition according to the present invention is formed by mixing the above information nucleic acid and additive such as a fat solubilizing agent, a heat stabilizer, a light stabilizer, a nucleic acid hydrolase inhibitor and/or a dispersing agent, and/or a combination of such additives. The additives may be formed into superfine particles so as to adsorb the information nucleic acid thereto or be bonded to the information nucleic acid with covalent bonds. With the thus formed information nucleic acid composition, the information nucleic acid can be dispersed in the product.

Besides, the information nucleic acid composition can be used in various states or properties and typically contained in paint, resin, fat, fiber and adhesive. In these cases, the information nucleic acid composition can be added to the raw material of the product in manufacturing process. The information nucleic acid composition may be contained in the product of fiber, leather, wood, paper and the like, in which the product is, for example, impregnated with or coated with the information nucleic acid composition.

### EXAMPLES

The present invention will be more readily understood with reference to the following Examples; however, these Examples are intended to illustrate the invention and are not to be construed to limit the scope of the invention.

### Example 1

(1) Synthesizing Information Nucleic Acid

To synthesize information DNA (primer binding site 1—identification information site—primer binding site 2) indicated below, nucleosides were sequentially bonded to each other by a phosphoramidite method.

5′-TGCACGCACCGTGTACTC-GGGATTAATTGGAGG-AGTGGAC
ACGTTGGTCGG-3′

(2) Preparation of Information Nucleic Acid Composition

The formed information nucleic acid synthesized above was adsorbed to zinc oxide having an average particle size of 0.02 μm to obtain DNA-containing fine particles. Thereafter, the DNA-containing fine particles were mixed with resin (plastic) at a ratio of 164 μg of the DNA-containing fine particles to 100 g of the resin, thereby obtaining an information nucleic acid composition.

(3) Method of Detection Carried Out by a Process Including the Following Steps:

(a) A test piece of the above information nucleic acid composition was finely fragmentized by using a cutter.

(b) 5 mL of sterilized distilled water was added to the fragmentized test piece and then stirred by using a magnetic stirrer, thereby extracting DNA in a layer of water.

(c) The layer of water was separated from the fragmentized test piece by using a centrifugal separator and then concentrated in a centrifugal evaporator to obtain a concentrated DNA solution.

(d) The concentrated DNA solution (5 μL), PCR buffer (5 μL), Taq polymerase (0.25 μL), sterilized distilled water (24.75 μL), primer 1 (5 μL), primer 2 (5 μL) and 2 mM dNTP (5 μL) were mixed together to obtain a mixed solution. The primers 1 and 2 had the following base sequences:

Primer 1 - - - 5′-TGCACGCACCGTGTACTC-3′
Primer 2 - - - 5′-CCGACCAACGTGTCCACT-3′

(e) The mixed solution was heated at 94° C. for 5 minutes and then subjected to repetition of 30 cycles of a temperature control which includes a heating at 94° C. for 30 seconds, a cooling at 40° C. for 30 seconds and a heating at 72° C. for 30 seconds in the order mentioned.

(f) The mixed solution was heated at 72° C. for 7 minutes and then preserved at 4° C.

(g) By using a single-stranded DNA splitting enzyme (S1 nuclease), excessive primers were split or decomposed. Thereafter, gel filtration was carried out to remove the split primers so as to refine the target double-stranded information DNA.

(g) 2, 3-dideoxynucleosidetriphosphate provided with fluorescence and one kind of primer (the above primer 1) were mixed to the refined information DNA to obtain a mixture information DNA.

(h) The mixture information DNA was heated at 94° C. for 5 minutes and then subjected to the repetition of 30 cycles of the temperature control as same as in the step (e).

(i) The mixture information DNA was refined by gel filtration and then supplied to an automatic sequencer, so that the base sequence of the information DNA was determined.

### Example 2

Procedure of Example 1 was repeated to determine the base sequence of an information DNA with the exception that an information DNA shown below was used to obtain an information nucleic acid composition, and the primer 2 at the step (d) was replaced with a primer shown below.

The information DNA:

5′-TGCACGCACCGTGTACTC-GGGATCACAAGGAGG-AGTGGA
CACGAAGGTCGG-3′

The primer 2 at the step (d):

5′-CCGACCTTCGTGTCCACT-3′

As appreciated from the above, according to the present invention, the target products can be authenticated individually by determining the base sequence of the information nucleic acid contained therein, even if they are mass-produced products such as industrial products.

The entire contents of Japanese Patent Applications P2004-286704 (filed Sep. 30, 2004) are incorporated herein by reference.

Although the invention has been described above by reference to certain embodiments and examples of the invention, the invention is not limited to the embodiments and examples described above. Modifications and variations of the embodiments and examples described above will occur to those skilled in the art, in light of the above teachings. The scope of the invention is defined with reference to the following claims.

What is claimed is:

1. An information nucleic acid comprising:
a base sequence portion having an arbitrary and known base sequence.

2. An information nucleic acid as claimed in claim 1, wherein the base sequence is a portion used for amplification of the nucleic acid.

3. An information nucleic acid as claimed in claim 1, further comprising an identification information site for improving authentication of individuality of the information nucleic acid.

4. An information nucleic acid as claimed in claim 1, wherein the base sequence is obtained by synthesis.

5. An information nucleic acid as claimed in claim 2, wherein the portion used for amplification has primer binding sites required for polymerase chain reaction (PCR), at both ends of the base sequence portion.

6. An information nucleic acid as claimed in claim 5, wherein number of bases in each of the primer binding sites is not less than 5.

7. An information nucleic acid as claimed in claim 5, wherein number of bases in each of the primer binding sites is not larger than 100.

8. An information nucleic acid as claimed in claim 1, wherein number of bases in whole of the information nucleic acid is not larger than 200.

9. An information nucleic acid as claimed in claim 1, wherein the base sequence has thymines, wherein a thymine and another thymine are not adjacent to each other in the base sequence.

10. An information nucleic acid as claimed in claim 1, further comprising a protective group for stabilizing the information nucleic acid.

11. An information nucleic acid as claimed in claim 10, wherein the information nucleic acid has a group at at least one of positions 5′ and 3′, the group being derivatized with the protective group.

12. An information nucleic acid as claimed in claim 10, wherein the protective group is at least one selected form the group consisting of a phosphoric ester group, an acyl group, an alkoxycarbonyl group, a benzyl group, a substituted benzyl group and an allyl group.

13. An information nucleic acid as claimed in claim 10, wherein the information nucleic acid has a hydroxyl group at position 5′, the hydroxyl group being derivatized with a biotin or a fluorescent molecule.

14. An information nucleic acid composition comprising:
an information nucleic acid including a base sequence portion having an arbitrary and known base sequence; and
an additive including at least one selected from the group consisting of a fat solubilizing agent, a heat stabilizer, a light stabilizer, a nucleic acid hydrolase inhibitor and a dispersing agent.


SEQUENCE LISTING (5 sequences)

SEQ ID NO: 1 - DNA, 51 bp, Artificial Sequence
  Description of Artificial Sequence Synthetic oligonucleotide
tgcacgcaccgtgtactcgggattaattggaggagtggacacgttggtcgg

SEQ ID NO: 2 - DNA, 18 bp, Artificial Sequence
  Description of Artificial Sequence Synthetic primer
tgcacgcaccgtgtactc

SEQ ID NO: 3 - DNA, 18 bp, Artificial Sequence
  Description of Artificial Sequence Synthetic primer
ccgaccaacgtgtccact

SEQ ID NO: 4 - DNA, 51 bp, Artificial Sequence
  Description of Artificial Sequence Synthetic oligonucleotide
tgcacgcaccgtgtactcgggatcacaaggaggagtggacacgaaggtcgg

SEQ ID NO: 5 - DNA, 18 bp, Artificial Sequence
  Description of Artificial Sequence Synthetic primer
ccgaccttcgtgtccact